(19) United States
(12) Patent Application Publication
(10) Pub. No.: US 2016/0046994 A1
(43) Pub. Date: Feb. 18, 2016

(54) NOVEL CD177 HAPLOTYPES, THEIR ROLE IN HNA-2 DEFICIENCY, AND METHODS OF USING

(71) Applicant: Regents of the University of Minnesota, Minneapolis, MN (US)

(72) Inventors: Jianming Wu, Plymouth, MN (US); Yunfang Li, Edina, MN (US)

(21) Appl. No.: 14/825,510

(22) Filed: Aug. 13, 2015

Related U.S. Application Data
(60) Provisional application No. 62/036,816, filed on Aug. 13, 2014.

(51) Int. Cl. C12Q 1/68

(52) U.S. Cl. CPC C12Q 1/6883; C12Q 2600/172; C12Q 2600/156

(57) ABSTRACT

This disclosure describes a novel CD177 haplotype and its involvement in HNA-2 deficiency. Methods of determining the CD177 haplotype of an individual are provided.

### CROSS REFERENCE TO RELATED APPLICATIONS

This application claims the benefit of priority under 35 U.S.C. §119(e) to U.S. Application No. 62/036,816 filed Aug. 13, 2014.

### TECHNICAL FIELD

This disclosure generally relates to novel CD177 haplotypes and methods of using.

### BACKGROUND

Transfusion-related acute lung injury (TRALI) is a severe adverse reaction occurring in human blood transfusions and is the most common cause of transfusion-related death in the United States. TRALI is often caused by alloantibodies against neutrophil antigens such as human neutrophil antigen 2 (HNA-2). To date, the genetic mechanism of HNA-2 deficiency remains unknown, and it remains unclear whether and how CD177 gene polymorphisms (e.g., SNPs) contribute to the heterogeneous HNA-2 expression. Elucidation of the molecular genetics and basis of the HNA-2 deficiency is a prerequisite for the use of effective genetic tests in the diagnosis, prognosis, and therapeutic decisions in treating human diseases.

The present disclosure is the first to delineate the genetic mechanism of HNA-2 deficiency and identify the relevant haplotypes. This characterization of the HNA-2 genetics and corresponding haplotypes will facilitate the development of effective genetic and clinical diagnostic tools in human medicine.

### SUMMARY

This disclosure describes a novel CD177 haplotype and its involvement in HNA-2 deficiency. Methods of determining the CD177 haplotype of an individual are provided.

In one aspect, a method of determining the CD177 haplotype of an individual is provided. Such a method typically includes providing a biological sample from the individual; amplifying a CD177 nucleic acid in the biological sample using a pair of primer oligonucleotides to produce a CD177 amplification product, wherein the pair of primer oligonucleotides comprises SEQ ID NOs: 9 and 10; amplifying the CD177 amplification product with a pair of ORF-specific primer oligonucleotides comprising SEQ ID NOs:12 and 13 to produce an ORF amplification product of 893 bp if the ORF haplotype is present; amplifying the CD177 amplification product with a pair of STP-specific primer oligonucleotides comprising SEQ ID NOs: 14 and 15 to produce a STP amplification product of 1254 bp if the STP haplotype is present; thereby determining the CD177 haplotype of the individual. Such a method can determine whether the individual is homozygous for the open reading haplotype (ORF), homozygous for the stop codon haplotype (STP), or heterozygous.

In another aspect, a method of determining the CD177 haplotype of an individual is provided. Such a method typically includes providing a biological sample from the individual; amplifying a CD177 nucleic acid using at least one primer oligonucleotide in the presence of at least one probe oligonucleotide, wherein the at least one primer oligonucleotide is selected from the group consisting of SEQ ID NO:38 and SEQ ID NO:39, wherein the at least one probe oligonucleotide is selected from the group consisting of SEQ ID NO:40 and SEQ ID NO:41, wherein the at least one probe oligonucleotide is labeled with a first fluorescent moiety and a quencher moiety; detecting the presence or absence of fluorescence emitted from the first fluorescent moiety, thereby determining the CD177 haplotype of the individual. In one representative embodiment, fluorescence in the presence of the probe oligonucleotide comprising SEQ ID NO:40 indicates the stop codon haplotype and wherein fluorescence in the presence of the probe oligonucleotide comprising SEQ ID NO:41 indicates the open reading haplotype.

As described herein, an individual comprising a stop codon haplotype is prone to HNA-2 antigen formation. Also as described herein, an individual comprising a stop codon haplotype exhibits CD177 (or HNA-2) deficiency.

Further as described herein, an individual having a STP haplotype is prone to produce alloantibodies responsible for acute lung injury, alloimmune neutropenia, and bone marrow transplantation failure, whereas, on the other hand, an individual having an ORF haplotype is prone to develop acute lung injury and alloimmune neutropenia following receipt of blood products containing HNA-2 alloantibodies.

In some embodiments, the methods described herein further includes determining the nucleotide at position 997. Typically, a wild type guanine at position 997 is in linkage disequilibrium with stop codon haplotype and deletion of the guanine at position 997 is in linkage disequilibrium with the open reading haplotype.

Representative biological samples include, without limitation, blood, plasma, saliva, urine, epithelial cells (e.g., from a mouth swab), hair, bone marrow cells, biopsy tissues.

In still other aspects, additional methods are provided such as, without limitation, methods of determining if an individual is prone to HNA-2 antigen formation; methods of determining if an individual exhibits CD177 (or HNA-2) deficiency; and/or methods of determining if an individual is prone to acute lung injury.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1A is a schematic showing the structure of the CD177 gene and the pseudogene on chromosome 19q13.2. CD177 gene and the pseudogene are separated by 8407 nucleotides. The CD177 gene contains nine exons. The CD177 pseudogene is highly homologous to CD177 between exon 4 and 9. Vertical red arrow indicates the CNV assay probe location.

FIG. 1B are graphs demonstrating that CD177 expression varies in different donors. Characteristic light-scatter properties were used to identify neutrophils in flow cytometry. Neutrophils from different donors vary in the percentages of CD177 positive cells. The right panel shows the absence of CD177 expression on neutrophils from a donor.

FIG. 1C is a gel of the amplification products of full-length CD177 cDNA from CD177 deficient donors. All CD177 deficient donors expressed full-length CD177 mRNA as the full-length CD177 cDNAs (1411 bps) were detected with RT-PCR.

FIG. 2 is a schematic that identifies the novel CD177 coding SNPs and SNP haplotypes described herein. The CD177 cDNAs from 11 CD177 deficient donors contain five novel cSNPs (824G>C, 828A>C, 829A>T, 832G>A, and 841A>G) that form a previously unidentified haplotype or variant (824C/828C/829T/832A/841G). The CD177 cSNP 829A>T is a nonsense polymorphism that creates a translation stop codon at amino acid position 263 (Lysine→Stop codon change) in CD177 open reading frame. The upper row of tracers is the haplotype containing 829A allele (designated as ORF or open reading frame allele) while the lower row tracer shows the haplotype containing 829T allele (designated as STP or stop codon allele). The middle tracer was from a heterozygous donor.

FIG. 3A shows that CD177 genotypes were determined with genomic DNA sequence analysis. CD177 expression was examined with flow cytometry analysis. All 829T homozygous donors (829TT, N=9) do not express CD177 on their neutrophils. The percentages of CD177⁺ neutrophils from heterozygous donors (829AT, N=73) were significantly (P<0.0001) lower than those from 829A homozygous donors (829AA, N=212).

FIG. 3B is a Western blot of CD177 expression in whole blood leukocytes. Representative donors were shown. No CD177 protein could be detected in all 829T homozygous donors (829TT), while heterozygous donors (829AT) express much less CD177 as compared to the 829A homozygous donors (829AA).

FIG. 4A shows the sequencing analysis of cDNA clones from two CD177⁻ donors who were heterozygous for the SNP 829A>T. The upper tracer is the wild-type (allele 997G) CD177 cDNA clone sequence. The 997G allele is in linkage disequilibrium with SNP 829T (or STP) allele as demonstrated by the sequence of cDNA clones from two CD177 deficient donors. The lower tracer is the sequence of CD177 cDNA clone with 997G deletion (or 997Δ allele), which is in linkage disequilibrium with SNP 829A allele (or ORF) in CD177 cDNA clones. Those two ORF/STP heterozygous donors manifested as CD177 deficient phenotype had the combination of one chromosome carrying 997G deletion (997Δ) allele and the other carrying the 829T (or STP) allele.

FIG. 4B demonstrates that genomic DNA sequence analysis confirmed that the guanidine nucleotide deletion occurs at genomic level. The upper tracer shows the wild-type genomic DNA sequence with the 997G. The lower tracer is the CD177 genomic sequence of a heterozygous donor with one chromosome containing 997G deletion and the other containing the wild-type.

FIG. 5A demonstrates the expression of common CD177 SNP haplotypes or variants (CD177.1: 31L-204N-205R-348A. CD177.2: 31H-204N-205R-348A. CD177.3: 31L-204D-205M-348A. CD177.4: 31L-204N-205R-348T) in 293 cells. No significant expression differences were observed among four haplotypes consisting of four common non-synonymous cSNPs (SNP 134A>T, 652A>G, 656G>T, and 1084G>A), which encode the amino acid substitutions of 31His>Leu, 204Asn>Asp, 205Arg>Met, and 348Ala>Thr. All experiments were repeated at least three times.

FIG. 5B demonstrates that no significant differences were observed in the HNA-2 alloantibody binding to four common CD177 variants (CD177.1: 31L-204N-205R-348A. CD177.2: 31H-204N-205R-348A. CD177.3: 31L-204D-205M-348A. CD177.4: 31L-204N-205R-348T). All experiments were repeated at least three times.

FIG. 5C demonstrates that CD177 expression was absent in cells stably transfected with the CD177 expression constructs of the SNP 829T allele (CD177-STP) or 997G deletion mutation (CD177-997AG). The SNP 829A allele (or CD177-ORF) served as the positive control for CD177 expression. All experiments were repeated at least three times.

FIG. 5D demonstrates that cells stably transfected with the expression constructs containing either SNP 829T (CD177-STP) allele or 997G deletion (CD177-997AG) allele failed to react with HNA-2 alloantibodies. The SNP 829A allele (or CD177-ORF) served as the positive control for CD177 expression. All experiments were repeated at least three times.

FIG. 6A is a schematic showing primer locations of the single-reaction allele-specific PCR assay used to determine the genotypes of the CD177 SNP ORF or STP haplotypes.

FIG. 6B is a gel showing that the PCR products were separated on an agarose gel. The size of internal control DNA fragment of human growth hormone gene is 429 bps. The CD177 genotypes were determined by the sizes and species of the DNA fragments in a single reaction. ORF-allele produces a DNA fragment containing 893 bps. STP-allele produces a DNA fragment containing 1254 bps. The specificity and accuracy of the assay were validated by the perfect match (100%) in 294 human subjects genotyped by Sanger sequencing methodology.

FIG. 6C is a plot of the results of the CD177 TaqMan genotyping assay. CD177 genotypes of the STP/ORF variant are clustered in three populations. The STP homozygous genotype is on the upper left corner, the ORF/STP heterozygous genotype in the middle, and the ORF/ORF homozygous on the lower right corner. The assay was validated by a perfect match (100%) of genotypes obtained by TaqMan assay with those determined by direct sequencing analysis and single tube allele-specific PCR in 294 human subjects.

FIG. 7 is a graph showing experiments in 293 cells transfected with the indicated mutant sequences (CD177-STP, CD177-829T, or CD177-997AG) or the controls (vector or CD177-ORF). FIG. 7 demonstrates that the T nucleotide substitution at position 829 alone leads to the absence of CD177 expression.

### DETAILED DESCRIPTION

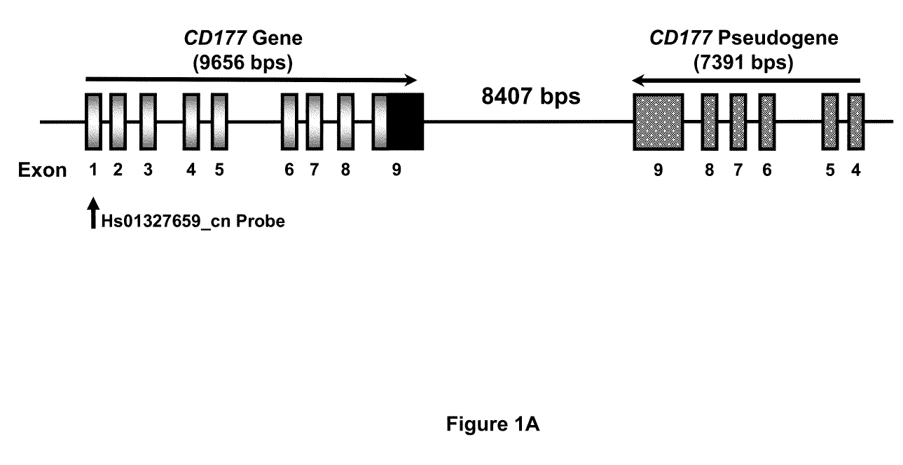

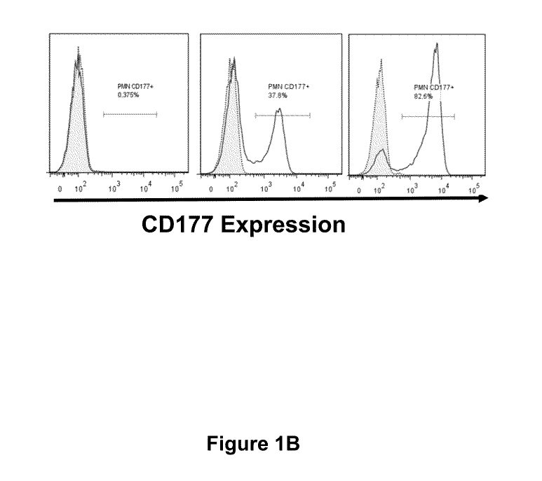

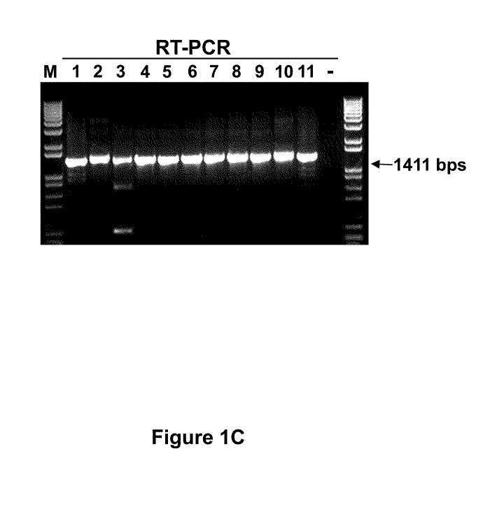

The HNA-2 antigen, also referred to as the NB 1 antigen, is encoded by the CD177 nucleic acid sequence, which contains nine exons (FIG. 1A). CD177 has an open reading frame of 1311 and encode 437 amino acids with a signal peptide of 21 residues. The CD177 nucleic acid sequence is expressed as a GPI-linked receptor with the mature peptide containing residues 22-408. A CD177 pseudogene is highly homologous to CD177 between exon 4 and 9 and is located 8.4-kb away from the CD177 gene on Chromosome 19 (FIG. 1A). Assays are described herein for determining the CD177 haplotype of an individual (e.g., an ORF/ORF homozygote, an ORF/STP heterozygote, or a STP/STP homozygote).

Given the importance of CD177 as a biomarker, determining the CD177 haplotype of an individual can be indicative of one or more disease states. For example, a low percentage of CD177⁺ neutrophils are significantly associated with myelodysplastic syndrome and chronic myelogenous leukemia, and CD177 deficiency also is a risk factor for acute myeloid leukemia. Therefore, the CD177 STP haplotype described herein may be a genetic risk factor for myelodysplastic syndrome, chronic myelogenous leukemia, acute myeloid leukemia, or myeloproliferative disorders. On the other hand, over-expression of CD177 is associated with PRV, inflammation due to, for example, severe sepsis or burns, myeloproliferative disorders (e.g., polycythemia vera, essential thromobocythemia, idiopathic myelofibrocythemia, and hypereosinophilic syndrome) and in healthy subjects after G-CSF treatment. In addition, overexpression of CD177 is a prognostic marker for gastric cancer and an indicator of increased erythropoietic activity in thalamassimia syndromes (e.g., beta-thalassemia). Therefore, the CD177 ORF haplotype described herein, particularly in homozygotes, may be a genetic risk factor for any of the above. Lastly, HNA-2-deficient individuals are prone (e.g., have an increased likelihood, have an inclination or tendency) to generate HNA-2 alloantibodies, via formation of HNA-2 antigens, after blood transfusions or bone marrow transplantation, or during or after pregnancy. Those alloantibodies against HNA-2 will lead to detrimental immune reactions, which have been shown to cause transfusion related acute lung injury, bone marrow transplantation failure, neonatal neutropenia, alloimmune neutropenia, autoimmune neutropenia, bone marrow transplantation failure, and drug-induced immune neutropenia.

### Nucleic Acids and Oligonucleotides

Primers and probes are provided herein that can be used to amplify and evaluate CD177 nucleic acid molecules. Primers that amplify a CD177 nucleic acid molecule can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 15 to 30 (e.g., 16, 18, 20, 21, 22, 23, 24, or 25) nucleotides in length.

Designing oligonucleotides to be used as hybridization probes can be performed in a manner similar to the design of primers. Probe oligonucleotides can be designed to hybridize to targets that contain a polymorphism (e.g., SNP) or a mutation, thereby allowing differential detection of CD177 haplotypes based on, for example, differential hybridization of probe oligonucleotides (e.g., a first probe oligonucleotide corresponding to a wild type sequence and a second probe corresponding to a polymorphic sequence). As with oligonucleotide primers, oligonucleotide probes usually have similar melting temperatures, and the length of each probe must be sufficient for sequence-specific hybridization to occur but not so long that fidelity is reduced during synthesis. Oligonucleotide probes are generally 15 to 30 (e.g., 16, 18, 20, 21, 22, 23, 24, or 25) nucleotides in length.

As used herein, constructs include vectors containing a CD177 nucleic acid molecule. Constructs or vectors suitable for use in the methods described herein are commercially available and/or produced by recombinant nucleic acid technology methods routine in the art. CD177 nucleic acid molecules can be obtained, for example, by chemical synthesis, direct cloning, or by PCR amplification. A CD177 nucleic acid molecule or fragment thereof can be operably linked to a promoter or other regulatory element such as an enhancer sequence, a response element, or an inducible element that modulates expression of the CD177 nucleic acid molecule. As used herein, operably linking refers to connecting a promoter and/or other regulatory elements to a CD177 nucleic acid molecule in such a way as to permit and/or regulate expression of the CD177 nucleic acid molecule. A promoter that does not normally direct expression of CD177 can be used to direct transcription of a CD177 nucleic acid using, for example, a viral polymerase, a bacterial polymerase, or a eukaryotic RNA polymerase II. Alternatively, a CD177 native promoter can be used to direct transcription of a CD177 nucleic acid. In addition, operably linked can refer to an appropriate connection between a CD177 promoter or regulatory element and a heterologous coding sequence (i.e., a non-CD177 coding sequence, for example, a reporter gene) in such a way as to permit expression of the heterologous coding sequence.

Constructs suitable for use in the methods described herein typically include, in addition to a CD177 nucleic acid molecule, sequences encoding a selectable marker (e.g., an antibiotic resistance gene) for selecting desired constructs and/or transformants, and an origin of replication. The choice of construct or vector systems usually depends upon several factors, including, but not limited to, the choice of host cells, replication efficiency, selectability, inducibility, and the ease of recovery.

Constructs containing a CD177 nucleic acid molecule can be propagated in a host cell using methods well known in the art. As used herein, the term host cell is meant to include prokaryotes and eukaryotes such as yeast, plant and animal cells. Prokaryotic hosts may include E. coli, Salmonella typhimurium, Serratia marcescens and Bacillus subtilis. Eukaryotic hosts include yeasts such as S. cerevisiae, S. pombe, Pichia pastoris, mammalian cells such as COS cells or Chinese hamster ovary (CHO) cells, insect cells, and plant cells such as Arabidopsis thaliana and Nicotiana tabacum. A construct or vector can be introduced into a host cell using any of the techniques commonly known to those of ordinary skill in the art. For example, calcium phosphate precipitation, electroporation, heat shock, lipofection, microinjection, and viral-mediated nucleic acid transfer are common methods for introducing nucleic acids into host cells. In addition, naked DNA can be delivered directly to cells (see, e.g., U.S. Pat. Nos. 5,580,859 and 5,589,466).

### Polymerase Chain Reaction (PCR)

U.S. Pat. Nos. 4,683,202, 4,683,195, 4,800,159, and 4,965,188 disclose conventional polymerase chain reaction (PCR) techniques. PCR typically employs two oligonucleotide primers that bind to a selected nucleic acid template (e.g., DNA or RNA). Pairs of primers useful for the methods provided herein include oligonucleotides capable of acting as points of initiation of nucleic acid synthesis within a CD177 nucleic acid sequence (e.g., SEQ ID NOs: 1 and 2, 7 and 8, 9 and 10, 12 and 13, 14 and 15, 12 and 15, 16 and 17, 18 and 19, or 38 and 39). A primer can be purified from a restriction digest by conventional methods, or it can be produced synthetically. The primer is preferably single-stranded for maximum efficiency in amplification, but the primer can be double-stranded. Double-stranded primers are first denatured, i.e., treated to separate the strands. One method of denaturing double stranded nucleic acids is by heating.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T. ruber, T. thermophilus, T. aquaticus, T. lacteus, T. rubens, Bacillus stearothermophilus, and Methanothermus fervidus. Nonetheless, polymerases that are not thermostable also can be employed in PCR assays provided the enzyme is replenished.

If the template nucleic acid is double-stranded, it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed for about 30 seconds to 4 minutes (e.g., 1 minute to 2 minutes 30 seconds, or 1.5 minutes).

If the double-stranded template nucleic acid is denatured by heat, the reaction mixture is allowed to cool to a temperature that promotes annealing of each primer to its target sequence on the template nucleic acid. The temperature for annealing is usually from about 35° C. to about 65° C. (e.g., about 40° C. to about 60° C.; about 45° C. to about 50° C.). Annealing times can be from about 10 seconds to about 1 minute (e.g., about 20 seconds to about 50 seconds; about 30 seconds to about 40 seconds). The reaction mixture is then adjusted to a temperature at which the activity of the polymerase is promoted or optimized, i.e., a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 40° to 80° C. (e.g., about 50° C. to about 70° C.; about 60° C.). Extension times can be from about 10 seconds to about 5 minutes (e.g., about 30 seconds to about 4 minutes; about 1 minute to about 3 minutes; about 1 minute 30 seconds to about 2 minutes).

PCR assays can employ CD177 template nucleic acid such as RNA or DNA (cDNA). The template nucleic acid need not be purified; it may be a minor fraction of a complex mixture, such as nucleic acid contained in human cells or in a biological sample. Nucleic acids may be extracted from a biological sample by routine techniques such as those described in Diagnostic Molecular Microbiology: Principles and Applications (Persing et al. (eds), 1993, American Society for Microbiology, Washington D.C.) or U.S. Pat. No. 6,811,971. Nucleic acids can be obtained from any number of sources, such as plasmids, or natural sources including bacteria, yeast, viruses, organelles, or higher organisms such as plants or animals.

The oligonucleotide primers (e.g., generally, pairs of primers; e.g., SEQ ID NOs: 1 and 2, 7 and 8, 9 and 10, 12 and 13, 14 and 15, 12 and 15, 16 and 17, 18 and 19, or 38 and 39) can be combined with PCR reagents under reaction conditions that induce primer extension. For example, chain extension reactions generally include 50 mM KCl, 10 mM Tris-HCl (pH 8.3), 1.5 mM MgCl₂, 0.001% (w/v) gelatin, 0.5-1.0 μg denatured template DNA, 50 pmoles of each oligonucleotide primer, 2.5 U of Taq polymerase, and 10% DMSO. The reactions usually contain 150 to 320 μM each of dATP, dCTP, dTTP, dGTP, or one or more analogs thereof.

The newly synthesized strands form a double-stranded molecule that can be used in the succeeding steps of the reaction. The steps of strand separation, annealing, and elongation can be repeated as often as needed to produce the desired quantity of amplification products corresponding to the CD177 nucleic acid molecule. The limiting factors in the reaction are the amounts of primers, thermostable enzyme, and nucleoside triphosphates present in the reaction. The cycling steps (i.e., denaturation, annealing, and extension) are preferably repeated at least once. For use in detection, the number of cycling steps will depend, e.g., on the nature of the sample. If the sample is a complex mixture of nucleic acids, more cycling steps typically will be required to amplify the target sequence sufficient for detection. Generally, the cycling steps are repeated at least about 20 times, but may be repeated as many as 40, 60, or even 100 times.

### Fluorescence Resonance Energy Transfer (FRET)

FRET technology (see, for example, U.S. Pat. Nos. 4,996,143, 5,565,322, 5,849,489, and 6,162,603) is based on a concept that when a donor and a corresponding acceptor fluorescent moiety are positioned within a certain distance of each other, energy transfer takes place between the two fluorescent moieties that can be visualized or otherwise detected and/or quantitated. A donor and corresponding acceptor fluorescent moiety can be contained on a single probe oligonucleotide or a donor and corresponding acceptor fluorescent moiety each can be contained on a different probe that binds, for example, within no more than 5 nucleotides of each other on the same strand such that FRET can occur. This minimal degree of separation typically brings the respective fluorescent moieties into sufficient proximity such that FRET occurs, however, it is to be understood that other separation distances (e.g., 6 or more nucleotides) are possible provided the fluorescent moieties are appropriately positioned relative to each other. Representative probe oligonucleotides, whether for use alone or in a pair, include, for example, SEQ ID NOs:40 and 41. Upon hybridization of the oligonucleotide probe(s) to the amplification product at the appropriate position(s), a FRET signal is generated. Hybridization temperatures can range from about 35° C. to about 65° C. (e.g., about 40° C. to about 60° C.; about 45° C. to about 55° C.; about 50° C.) for about 10 seconds to about 1 minute (e.g., about 20 seconds to about 50 seconds; about 30 seconds to about 40 seconds).

Fluorescent analysis can be carried out using, for example, a photon counting epifluorescent microscope system (containing the appropriate dichroic mirror and filters for monitoring fluorescent emission at the particular range), a photon counting photomultiplier system, or a fluorometer. Excitation to initiate energy transfer can be carried out with an argon ion laser, a high intensity mercury (Hg) arc lamp, a fiber optic light source, or other high intensity light source appropriately filtered for excitation in the desired range.

As used herein with respect to donor and corresponding acceptor fluorescent moieties, “corresponding” refers to an acceptor fluorescent moiety having an emission spectrum that overlaps the excitation spectrum of the donor fluorescent moiety. The wavelength maximum of the emission spectrum of the acceptor fluorescent moiety should be at least 100 nm greater than the wavelength maximum of the excitation spectrum of the donor fluorescent moiety. Accordingly, efficient non-radiative energy transfer can be produced therebetween. It would be understood by the skilled artisan that the corresponding acceptor fluorescent moiety can be a quencher, which has the ability to absorb the wavelength from the donor fluorescent moiety.

Fluorescent donor and corresponding acceptor moieties are generally chosen for (a) high efficiency Förster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothiocyanatostilbene-2,2′-disulfonic acid, 7-diethylamino-3-(4′-isothiocyanatophenyl)-4-methylcoumarin, succinimidyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC™-Red 640, LC™-Red 705, Cy5, Cy5.5, Lissamine rhodamine B sulfonyl chloride, tetramethyl rhodamine isothiocyanate, rhodamine×isothiocyanate, erythrosine isothiocyanate, fluorescein, diethylenetriamine pentaacetate or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

The donor and acceptor fluorescent moieties can be attached to the appropriate probe oligonucleotide via a linker arm. The length of each linker arm is important, as the linker arms will affect the distance between the donor and corresponding acceptor fluorescent moieties. The length of a linker arm for the purpose of the methods provided herein is the distance in Angstroms (Å) from the nucleotide base to the fluorescent moiety. In general, a linker arm is from about 10 to about 25 Å (e.g., about 15 Å to about 20 Å). The linker arm may be of the kind described in WO 84/03285. WO 84/03285 also discloses methods for attaching linker arms to a particular nucleotide base, and also for attaching fluorescent moieties to a linker arm.

An acceptor fluorescent moiety such as an LC™-Red 640-NHS-ester can be combined with C6-Phosphoramidites (available from ABI (Foster City, Calif.) or Glen Research (Sterling, Va.)) to produce, for example, LC™-Red 640-Phosphoramidite. Frequently used linkers to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as fluorescein-CPG from BioGenex (San Ramon, Calif.)), or 3′-amino-CPG's that require coupling of a fluorescein-NHS-ester after oligonucleotide synthesis.

### Detection of CD177 Haplotypes

PCR methods can be used to detect the haplotype of a CD177 nucleic acid molecule from an individual. In some embodiments, a portion of a CD177 nucleic acid molecule is amplified from a biological sample using a pair of CD177 primer oligonucleotides (e.g., SEQ ID NOs:9 and 10). Each member of the pair of primer oligonucleotides anneals to a target within or adjacent to a CD177 nucleic acid molecule such that at least a portion of the resulting amplification product contains nucleic acid sequence corresponding to CD177. The amplification product then can be amplified with a pair of ORF-specific primer oligonucleotides (e.g., SEQ ID NOs:12 and 13) and/or a pair of STP-specific primer oligonucleotides (e.g., SEQ ID NOs:14 and 15) to determine the haplotype of the individual.

If the ORF haplotype is present, amplification using SEQ ID NOs:12 and 13 produces a 893 bp amplification product, whereas if the STP haplotype is present, amplification using SEQ ID NOs:14 and 15 produces a 1254 bp amplification product. The size (or molecular weight) of nucleic acids routinely is determined using, for example, agarose gel electrophoresis or mass spectroscopy (MS). Based on the amplification product(s) produced, the CD177 haplotype can be determined for the individual (e.g., ORF/ORF homozygote, ORF/STP heterozygote, or STP/STP homozygote).

In some embodiments, real-time PCR methods can be used to detect the haplotype of a CD177 nucleic acid molecule from an individual. In some embodiments, a portion of a CD177 nucleic acid molecule is amplified from a biological sample using a pair of CD177 primer oligonucleotides (e.g., SEQ ID NO: 38 and 39) and detected using at least one probe oligonucleotide (e.g., SEQ ID NO: 40 and 41) and a commercially-available real-time PCR instrumentation (e.g., ABI PRISM® 7700 Sequence Detection System, Applied Biosystems, Foster City, Calif.; or LIGHTCYCLER™, Roche Molecular Biochemicals, Indianapolis, Ind.). With real-time PCR, it is important that the amplification product contains the nucleic acid sequences that are complementary to at least one probe oligonucleotide.

As demonstrated herein, the nucleotide at position 997, relative to SEQ ID NO:42, is in linkage disequilibrium with the haplotypes described herein. For example, as demonstrated herein, a wild type guanine at position 997 is in linkage disequilibrium with the stop codon haplotype, while a deletion of the guanine at position 997 is in linkage disequilibrium with the open reading frame haplotype.

As used herein, “amplifying” refers to the process of synthesizing nucleic acid molecules that are complementary to one or both strands of a template nucleic acid molecule (e.g., a CD177 nucleic acid molecule). Amplifying a nucleic acid molecule typically includes denaturing the template nucleic acid, annealing primers to the template nucleic acid at a temperature that is below the melting temperatures of the primers, and enzymatically elongating from the primers to generate an amplification product. Amplification typically requires the presence of deoxyribonucleoside triphosphates, a DNA polymerase enzyme (e.g., PLATINUM® Taq) and an appropriate buffer and/or co-factors for optimal activity of the polymerase enzyme (e.g., MgCl₂ and/or KCl). As used herein, “hybridizing” refers to the annealing of at least one probe oligonucleotide to an amplification product. Hybridization conditions typically include a temperature that is below the melting temperature of the probe(s) but that avoids non-specific hybridization of the probe(s).

In a TAQMAN embodiment, at least one probe oligonucleotide is included in the reaction during amplification. For determining the presence or the absence of a SNP, either or both of two probes can be used; a first probe that is complementary to the wild type sequence and a second probe that is complementary to the polymorphic sequence. In a LIGHTCYCLER two-probe embodiment, two probes that bind in close proximity to one another within the amplification product are used, where one is labeled with a donor fluorescent moiety and the other is labeled with a corresponding acceptor fluorescent moiety. Given the proper design of the probe oligonucleotide(s), the presence or absence of FRET between the donor fluorescent moiety and the corresponding acceptor fluorescent moiety is determinative of the CD177 haplotype (e.g., ORF/ORF homozygote, ORF/STP heterozygote, or STP/STP homozygote).

Representative biological samples that can be used in practicing the methods described herein include, without limitation, blood, plasma, saliva, urine, epithelial cells (e.g., from a mouth swab), hair, bone marrow cells, or biopsy tissues. Collection and storage methods of biological samples are known to those of skill in the art. Biological samples can be processed (e.g., by nucleic acid extraction methods and/or kits known in the art) to release or isolate CD177 nucleic acid, or in some cases, the biological sample can be contacted directly with the PCR reaction components and the appropriate oligonucleotides. For example, nucleic acids can be extracted from biological samples using the commercially-available total nucleic acid kit, MagNA Pure (Roche Applied Science, Indianapolis, Ind.).

Melting curve analysis is an additional step that can be included in a cycling profile. Melting curve analysis is based on the fact that DNA melts at a characteristic temperature called the melting temperature (Tm), which is defined as the temperature at which half of the DNA duplexes have separated into single strands. The melting temperature of a DNA depends primarily upon its nucleotide composition. Thus, DNA molecules rich in G and C nucleotides have a higher Tm than those having an abundance of A and T nucleotides. By detecting the temperature at which signal is lost, the melting temperature of at least one probe oligonucleotide can be determined. Similarly, by detecting the temperature at which signal is generated, the annealing temperature of the at least one probe oligonucleotide can be determined. The melting temperature(s) of the at least one probe oligonucleotide from the amplification product can confirm the haplotype in the sample. For example, the presence of a particular CD177 haplotype in a sample can be confirmed by detecting a melting curve within an established temperature range.

Within each thermocycler run, control samples typically are cycled as well. Positive control samples can amplify nucleic acid control template (e.g., one or more known CD177 haplotypes) using, for example, control primers and control probes. Positive control samples can also amplify, for example, a plasmid construct containing a non-CD177 nucleic acid molecule. A control can be amplified internally (e.g., within the sample) or in a separate side-by-side sample. Each thermocycler run should also include a negative control that, for example, lacks CD177 template DNA. Such controls are indicators of the success or failure of the amplification, hybridization and/or FRET reaction. Therefore, control reactions can readily determine, for example, the ability of primers to anneal with sequence-specificity and to initiate elongation, as well as the ability of one or more probes to hybridize with sequence-specificity and for FRET to occur.

As described herein, amplification products can be detected using labeled hybridization probes that take advantage of FRET technology. There are a number of commercially available platforms, as discussed below. It is understood, however, that the present disclosure is not limited by the configuration of one or more commercially available instruments.

A representative FRET format utilizes TAQMAN® technology. TAQMAN® technology utilizes one single-stranded hybridization probe labeled with two fluorescent moieties. When a first fluorescent moiety is excited with light of a suitable wavelength, the absorbed energy is transferred to a second fluorescent moiety according to the principles of FRET. The second fluorescent moiety is generally a quencher molecule. During the annealing step of the PCR reaction, the labeled hybridization probe binds to the target nucleic acid (i.e., the amplification product) and is degraded by the 5′ to 3′ exonuclease activity of the Taq Polymerase during the subsequent elongation phase. As a result, the excited fluorescent moiety and the quencher moiety become spatially separated from one another. As a consequence, upon excitation of the first fluorescent moiety in the absence of the quencher, the fluorescence emission from the first fluorescent moiety can be detected. By way of example, an ABI PRISM® 7700 Sequence Detection System (Applied Biosystems, Foster City, Calif.) uses TAQMAN® technology, and is suitable for performing the methods described herein for determining a CD177 haplotype. Information on PCR amplification and detection using an ABI PRISM® 7700 system can be found at appliedbiosystems.com/products on the World Wide Web.

Molecular beacons in conjunction with FRET also can be used to detect the presence of a CD177 haplotype using the methods described herein. Molecular beacon technology uses a single hybridization probe oligonucleotide labeled at each end of the probe with a first fluorescent moiety and a second fluorescent moiety. Oftentimes, the second fluorescent moiety is a quencher. Molecular beacon technology uses a probe oligonucleotide having a sequence that permit secondary structure formation (e.g., a hairpin). As a result of secondary structure formation within the probe oligonucleotide, both fluorescent moieties are in spatial proximity when the probe is in solution. After hybridization to target nucleic acids (i.e., amplification products), the secondary structure of the probe oligonucleotide is disrupted and the fluorescent moieties become separated from one another such that after excitation with light of a suitable wavelength, the emission of the first fluorescent moiety can be detected.

Another common format of FRET technology utilizes two hybridization probe oligonucleotides. Each probe oligonucleotide can be labeled with a different fluorescent moiety and are generally designed to hybridize in close proximity to each other (e.g., within 5 nucleotides) in a target DNA molecule (e.g., an amplification product). A donor fluorescent moiety, for example, fluorescein, is excited at 470 nm by the light source of, for example, a LIGHTCYCLER™ Instrument. During FRET, the fluorescein transfers its energy to an acceptor fluorescent moiety such as LIGHTCYCLER™ Red 640 (LC™ Red 640) or LIGHTCYCLER™ Red 705 (LC™ Red 705). The acceptor fluorescent moiety then emits light of a longer wavelength, which is detected by the optical detection system of the instrument. Efficient FRET can only take place when the fluorescent moieties are in direct local proximity and when the emission spectrum of the donor fluorescent moiety overlaps with the absorption spectrum of the acceptor fluorescent moiety.

### Articles of Manufacture/Kits

This document further describes articles of manufacture that can be used to detect CD177 SNPs and/or haplotypes. An article of manufacture as described herein can include primers and probes used to detect CD177 haplotypes, together with suitable packaging materials. Representative primers and probes for detection of CD177 haplotypes are capable of hybridizing to CD177 nucleic acid molecules. Methods of designing primers and probes are disclosed herein, and representative examples of primers and probes that amplify and hybridize to CD177 nucleic acid molecules are provided (e.g., SEQ ID NOs: 1 and 2, 7 and 8, 9 and 10, 12 and 13, 14 and 15, 12 and 15, 16 and 17, 18 and 19, 38 and 39, 40 or 41).

Articles of manufacture as described herein also can include one or more fluorescent moieties for labeling the probes or, alternatively, the probes supplied with the kit can be labeled. For example, an article of manufacture may include a donor fluorescent moiety and an acceptor fluorescent moiety for labeling one or more probes. Examples of suitable FRET donor fluorescent moieties and corresponding acceptor fluorescent moieties are provided above.

Articles of manufacture also can contain a package insert or package label having instructions thereon for using the primers and probes to detect a CD177 haplotype in a biological sample. Articles of manufacture may additionally include reagents for carrying out the methods disclosed herein (e.g., buffers, polymerase enzymes, co-factors, or agents to prevent contamination). Such reagents may be specific for one of the commercially available instruments described herein.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### Study Subjects

Healthy blood donors were recruited at the Memorial Blood Center (mbc.org/Home on the World Wide Web) in St. Paul, Minn. The age of healthy control donors ranges from 19 to 84 years-old. The human study was approved by the Institutional Review Board for Human Use at the University of Minnesota.

### Example 2

### Evaluation of CD177 (HNA-2) Expressions on Neutrophils

To determine the expression of HNA-2 and the percentage of HNA-2 positive neutrophils in different blood donors, 100 μl fresh whole blood samples were stained with either FITC-conjugated anti-human CD177 mAb (clone MEM166, mIgG1, Thermo Scientific) or FITC-conjugated mIgG1 isotype control in separate tubes. After staining, blood samples were treated with 1×FACS Lysing Solution (BD Biosciences) to lyse the red blood cells, followed by analysis on a FACS Canto flow cytometer (BD Biosciences). The collected flow cytometry data were analyzed using FlowJo software (Tree Star Inc., flojo.com on the World Wide Web). Characteristic light-scatter properties were used to identify neutrophils in flow cytometry. HNA-2 deficient donors were identified using previously published methods (Moritz et al., 2010, Vox. Sang., 98:160-66). HNA-2 deficient donors were defined to have less than 5% of granulocytes positive for MEM-166.

### Example 3

### Western Blot Analysis of CD177 Protein

Peripheral blood leukocytes (2×10⁷ cells) were lysed in phosphate-buffered saline containing 1% Nonidet P-40 with protease inhibitor cocktail (Roche Applied Science, Indianapolis, ID) for 1 hr on ice. The cell lysates were centrifuged at 12000×g for 10 min and the supernatants were harvested for protein quantification. The equal amount of total proteins (50 μg) from each donor was separated on a non-reducing SDS-PAGE gel and then transferred to a nitrocellulose membrane. Western blotting analysis was carried out with mouse anti-CD177 mAb (clone MEM166, Thermo Scientific) and rabbit anti-actin mAb (LI-COR Biosciences, Lincoln, Nebr.). IRDye 800CW labeled goat anti-mouse and IRDye 600 labeled goat anti rabbit antibodies were used for imaging analysis on an Odyssey Infrared Imager according to vendor's instructions (LI-COR Biosciences).

### Example 4

### Nucleic Acid Isolation

Human genomic DNA was isolated from EDTA anti-coagulated peripheral blood using the Puregene DNA isolation kit (Gentra Systems, Minneapolis, Minn.) by following the vendor's instruction. Total RNA was purified from peripheral blood leukocytes using TRIzolTM total RNA isolation reagent (Invitrogen, Carlsbad, Calif.).

### Example 5

### Determination of CD177 CNVs

The CNVs of CD177 were determined using TaqMan® Copy Number Assay kit (Assay ID Hs01327659_cn) (FIG. 1A) with the probe targeting the exon 1 of CD177 (Applied Biosystems, Foster City, Calif., USA) and with RNase P (4403326, VIC-TAMRA dual-labeled probe; Applied Biosystems) as the reference assay. Duplex quantitative real-time PCR reactions were carried out on an Applied Biosystems 7500 Real-Time PCR System according to the manufacturer's instructions. All samples were tested in duplicates, and fluorescence signals were normalized to ROX. TaqMan assay quantitative PCR amplification curves were analyzed using 7500 Software on a plate by plate basis, and the CN was assigned from the raw Cq values using CopyCaller™ software (version 2.0; Applied Biosystems).

### Example 6

### RT-PCR and cDNA Sequencing

Five μg of total RNA was used for cDNA synthesis with the SuperScript™ pre-amplification system (Invitrogen). The 1411-bp cDNA fragment covering the entire CD177 coding region was amplified with RT-PCR using sense primer (5′-CTG AAA AAG CAG AAA GAG ATT ACC AGC CAC AG-3′ (SEQ ID NO:1)) and anti-sense primer (5′-GTC CAA GGC CAT TAG GTT ATG AGG TCA GA-3′ (SEQ ID NO:2)). The PCR reaction was performed with 2 μl of cDNA, 200 nM of each primer, 200 μM of dNTPs, 2.0 mM of MgSO₄, and 1 U of Platinum® Taq DNA polymerase High Fidelity (Invitrogen) in a 25 μl reaction volume. The ABI Veriti 96-well Thermal Cycler was used for the PCR reaction starting with 94° C. for 3 min, 35 cycles of denaturing at 94° C. for 30 sec, annealing at 56° C. for 45 sec, extension at 68° C. for 1 min and 30 sec with a final extension at 72° C. for 7 min. All the PCR products, treated with ExoSAP-IT (Affymetrix, Santa Clara, Calif.) to remove unincorporated primers and nucleotides according to the manufacturer's instruction, were assessed by direct Sanger sequencing on an ABI 3730×1 DNA Analyzer with BigDye v3.1 Sequencing kit (Applied Biosystems, Inc., Foster City, Calif.). CD177 cDNA was also directly cloned into pCR2.1-TOPO vector (Invitrogen, Carlsbad, Calif.). Multiple clones containing CD177 cDNA were selected and subsequently sequenced to confirm CD177 SNPs. Two sense primers (5′-A CCT TGA GGT GCC CAG TCT GCT T-3′ (SEQ ID NO:3) and 5′-ACC GGC AGT GTC CTA CCT GTG T-3′ (SEQ ID NO:4)) and two antisense primers (5′-AAG CAG ACT GGG CAC CTC AAG G-3′ (SEQ ID NO:5) and 5′-ACA CAG GTA GGA CAC TGC CGG T-3′ (SEQ ID NO:6)) were used to sequence the full-length CD177 cDNA coding region. The electropherogram data, aligned by the DNASTAR software (DNAStar, Madison, Wis.) were used for the identification of gene polymorphisms.

### Example 7

### Genomic DNA Sequence Analysis of CD177 Gene

Since CD177 and the pseudogene contain a highly homologous region between exon 4 and 9 (Bettinotti et al., 2002, Clin. Immunol., 102:138:44; Caruccio et al., 2006, Transfusion, 46:441-7) (FIG. 1A), we used the long-template PCR strategy to obtain the CD177-specific products for sequence analyses. Long-template PCR was carried out to amplify the CD177 genomic DNA containing all 9 exons using a sense primer (5′-CTG AAA AAG CAG AAA GAG ATT ACC AGC CAC AG-3′ (SEQ ID NO:7)) and antisense primer (5′-GTC CAA GGC CAT TAG GTT ATG AGG TCA GA-3′ (SEQ ID NO:8)). The PCR reaction was performed with 200 ng DNA, 200 nM of each primer, 200 μM of dNTPs, 2.0 mM of MgSO₄, and 2 U of Platinum® Taq DNA polymerase High Fidelity (Invitrogen) in a 25 μl reaction volume. The ABI Veriti 96-well Thermal Cycler was used for the PCR reaction starting with 95° C. for 3 min; 10 cycles of denaturing at 94° C. for 30 sec, annealing at 64° C. for 30 sec, extension at 68° C. for 8 min and 30 sec; 30 cycles of denaturing at 94° C. for 30 sec, annealing at 54° C. for 30 sec, extension at 68° C. for 8 min and 30 sec; with a final extension at 68° C. for 5 min. The CD177 PCR DNA fragment (8728 base pairs), treated with ExoSAP-IT (Affymetrix, Santa Clara, Calif.) to remove unincorporated primers, were assessed by direct Sanger sequencing on an ABI 3730×1 DNA Analyzer with BigDye v3.1 Sequencing kit with a sequencing primer (5′-TCT TTG CCC CAC ACT AAA CA-3′ (SEQ ID NO:11)) annealing to the intron 6. Sequence data analysis was performed using LaserGene DNAstar software to determine genotypes. Table 1 lists all the primers used in the current genetic study.

### Example 8

### Generation of CD177 Expression Constructs

The human CD177 expression constructs were generated by cloning Hind III/Xba I-flanked RT-PCR products containing full-length CD177 coding region (nucleotide position 25 to 1419, GenBank Accession No. NM 020406.2, see the nucleic acid sequence shown in SEQ ID NO:42, which encodes the amino acid sequence of SEQ ID NO:43) into the eukaryotic expression vector pcDNA3 (Gibco BRL). The Hind III/Xba I-flanked CD177 cDNA was amplified from the synthesized cDNA of a blood donor using the upper primer 5′-CCC AAG CTT ACC AGC CAC AGA CGG GTC ATG AG-3′ (underlined and bold nucleotides are Hind III cutting site; SEQ ID NO:34) and the lower primer 5′-TGC TCT AGA GAG GTC AGA GGG AGG TTG AGT GTG-3′ (underlined and bold nucleotides are Xba I cutting site; SEQ ID NO:35). The changes at nucleotide position 134, 652, 656, 824, 828, 829, 832, 841, 997, and 1084 were generated on the expression constructs using QuikChange Site-Directed mutagenesis kit (Stratagene, La Jolla, Calif.) and the primer sets listed in Table 4.

(SEQ ID NO: 42)
ctgctgaaaa agcagaaaga gattaccagc cacagacggg
tcatgagcgc ggtattactg ctggccctcc tggggttcat
cctcccactg ccaggagtgc aggcgctgct ctgccagttt
gggacagttc agcatgtgtg gaaggtgtcc gacctgcccc
ggcaatggac ccctaagaac accagctgcg acagcggctt
ggggtgccag gacacgttga tgctcattga gagcggaccc
caagtgagcc tggtgctctc caagggctgc acggaggcca
aggaccagga gccccgcgtc actgagcacc ggatgggccc
cggcctctcc ctgatctcct acaccttcgt gtgccgccag
gaggacttct gcaacaacct cgttaactcc ctcccgcttt
gggccccaca gcccccagca gacccaggat ccttgaggtg
cccagtctgc ttgtctatgg aaggctgtct ggaggggaca
acagaagaga tctgccccaa ggggaccaca cactgttatg
atggcctcct caggctcagg ggaggaggca tcttctccaa
tctgagagtc cagggatgca tgccccagcc agtttgcaac
ctgctcaatg ggacacagga aattgggccc gtgggtatga
ctgagaactg cgatatgaaa gattttctga cctgtcatcg
ggggaccacc attatgacac acggaaactt ggctcaagaa
cccactgatt ggaccacatc gaataccgag atgtgcgagg
tggggcaggt gtgtcaggag acgctgctgc tcctagatgt
aggactcaca tcaaccctgg tggggacaaa aggctgcagc
actgttgggg ctcaaaattc ccagaagacc accatccact
cagcccctcc tggggtgctt gtggcctcct atacccactt
ctgctcctcg gacctgtgca atagtgccag cagcagcagc
gttctgctga actccctccc tcctcaagct gcccctgtcc
caggagaccg gcagtgtcct acctgtgtgc agccccttgg
aacctgttca agtggctccc cccgaatgac ctgccccagg
ggcgccactc attgttatga tgggtacatt catctctcag
gaggtgggct gtccaccaaa atgagcattc agggctgcgt
ggcccaacct tccagcttct tgttgaacca caccagacaa
atcgggatct tctctgcgcg tgagaagcgt gatgtgcagc
ctcctgcctc tcagcatgag ggaggtgggg ctgagggcct
ggagtctctc acttgggggg tggggctggc actggcccca
gcgctgtggt ggggagtggt ttgcccttcc tgctaactct
attaccccca cgattcttca ccgctgctga ccacccacac
tcaacctccc tctgacctca taacctaatg gccttggaca
ccagattctt tcccattctg tccatgaatc atcttcccca
cacacaatca ttcatatcta ctcacctaac agcaacactg
gggagagcct ggagcatccg gacttgccct atgggagagg
ggacgctgga ggagtggctg catgtatctg ataatacaga
ccctgtcctt tctcccagtg ctgggatttc tccatgtgag
ggggcagcag gacacccagg gatctagcgt gggggaggag
aggagcctaa tgagaaaatg accatctaaa gcctgccctt
cattggtctg gttcacgtct ccaaaccagc ttggatggta
gcagagactt cagggtgctc cagccaaacg tatttgggca
tcaccatgac ctgggagggg aagatgcact gagacgtatg
aggcttccag cctagcagcc agggccctag cacaaacagg
aggctcgccc catctgagca actgcaggag aggttagtac
agtcatgcat tgcttaacga cagggacgtg tcgttagaaa
tgtgtcgtta ggtgatttta tgaccatagg aacattgtag
cgtgcactta caccaaccca gatggtacag cccaatacac
acccaggatg gacgctagag tcgactgctc ctaggctaca
agcctgcagt gcatgttatg gtgtgaatac tgcaggcaat
cttaacacca cggcaagtat ttgtgcatct acacacatct
aaacatagaa aaggtacagc ataaatacac tattgtcatc
tcagcagacc accgttctat acgcaattcg tcgctgaccc
aaacgttgct atgtagcatc tgcgtatcgt gggataattg
acatgagggc ttgagagaac tccagaaaaa aatgggttag
cattttccca gagctgttat cattgggtct ctcttaccac
cata
(SEQ ID NO: 43)
MSAVLLLALLGFILPLPGVQALLCQFGTVQHVWKVSDLPRQWTPKNTSCD
SGLGCQDTLMLIESGPQVSLVLSKGCTEAKDQEPRVTEHRMGPGLSLISY
TFVCRQEDFCNNLVNSLPLWAPQPPADPGSLRCPVCLSMEGCLEGTTEEI
CPKGTTHCYDGLLRLRGGGIFSNLRVQGCMPQPVCNLLNGTQEIGPVGMT
ENCDMKDFLTCHRGTTIMTHGNLAQEPTDWTTSNTEMCEVGQVCQETLLL
LDVGLTSTLVGTKGCSTVGAQNSQKTTIHSAPPGVLVASYTHFCSSDLCN
SASSSSVLLNSLPPQAAPVPGDRQCPTCVQPLGTCSSGSPRMTCPRGATH
CYDGYIHLSGGGLSTKMSIQGCVAQPSSFLLNHTRQIGIFSAREKRDVQP
PASQHEGGGAEGLESLTWGVGLALAPALWWGVVCPSC

The full-length CD177 cDNAs were also cloned into a pMX-PIE vector that contains a puromycine resistance gene. The BamH I/EcoR I-flanked CD177 cDNA inserts were amplified from CD177/pcDNA3 expression constructs using the upper primer 5′-CCC GGA TCC ACC AGC CAC AGA CGG GTC ATG AG-3′ (underlined and bold nucleotides are BamH I cutting site; SEQ ID NO:36) and the lower primer 5′-TGC GAA TTC GAG GTC AGA GGG AGG TTG AGT GTG-3′ (underlined and bold nucleotides are EcoR I cutting site; SEQ ID NO:37). CD177 cDNA inserts digested with BamH I and EcoR I were subsequently cloned into the pMX-PIE vector as a bi-cistronic coding unit containing CD177 cDNA, followed by an internal ribosomal entry site (IRES) element and the enhanced green fluorescent protein (EGFP). All expression constructs containing human CD177 cDNA inserts were confirmed by Sanger sequencing.

### Example 9

### Generation of Cell Lines Expressing CD177

The 293 cells (human embryonic kidney cell line) from ATCC (ATCC#CRL-1573, Manassas, Va.) were maintained in the DMEM medium supplemented with 10% fetal calf serum and L-glutamine (2 mM) in 5% CO₂). Transfection reactions were carried out in the 100 mm cell culture dishes with the plasmid DNA (20 μg) purified with OMEGA Plasmid Maxi Kit (Omega Bio-Tek, Norcross, Ga.) and 40 μl of Lipofectamine 2000 reagent (Invitrogen). Transfected cells were cultured in DMEM medium supplemented with 10% fetal calf serum for two days before harvesting the cells for CD177 expression or the selection of stable cell lines with the supplement of G418 (final concentration: 1 mg/ml). The polyclonal cells surviving the G418 selection were sorted with Stemcell EasySep Cell Sorter for equal CD177 expression. The CD177/pMX-PIE constructs were also transfected into 293 cells. Forty-eight hours after transfection, cells were selected in medium containing puromycin (1.5 μg/ml) for two weeks. The stable cell lines survived the puromycin selection were used for flow cytometry to examine CD177 expression.

### Example 10

### Evaluation of CD177 Expressions on the Transfected Cells

To determine the expression of CD177 on the transfected 293 cells, 5×10⁵ cells in 100 μl of PBS were stained with either FITC-conjugated or APC-conjugated anti-human CD177 mAb (clone MEM166, mIgG1, Thermo Scientific). After washing twice with PBS, cells were analyzed on a FACS Canto flow cytometer (BD Biosciences). Five defined HNA-2 alloantibodies from the American Red Cross Neutrophil Serology Laboratory were also used to evaluate the binding of HNA-2 to the cell lines expressing CD177 variants. To determine the reactivity of HNA-2 alloantibody to the transfected 293 cells, 5×10⁵ cells were incubated in 100 μl of PBS containing HNA-2 serum samples (1:500 dilutions) for 30 min at room temperature. After washing twice with PBS, cells were stained with FITC-conjugated goat anti-human IgG and then analyzed on a FACS Canto flow cytometer. The collected flow cytometry data were analyzed using FlowJo software.

### Example 11

### CD177 SNP Genotyping Assays

To facilitate genetic tests, a single-reaction allele-specific PCR assay was designed to genotype the CD177 SNP haplotypes (ORF or STP allele). Four CD177 primers (two primers for ORF-specific reaction and two primers for STP-specific reaction) plus two primers for human growth hormone gene as the internal control (Table 1) were used in a single PCR amplification. The PCR reaction was performed with 20 ng DNA, 200 nM of each primer, 200 μM of dNTPs, 1.5 mM of MgCl₂, and 1 U of Taq DNA polymerase in a 25 μl reaction volume. The ABI Veriti 96-well Thermal Cycler was used for the PCR reaction starting with 95° C. for 3 min; 35 cycles of denaturing at 94° C. for 20 sec, annealing at 58° C. for 30 sec, extension at 72° C. for 1 min and 20 sec; with a final extension at 72° C. for 7 min. Agarose gels (1.5%) were used to visualize and estimate the sizes of DNA fragments.

The high-throughput TaqMan assay could not directly be used for CD177 exon 7 SNPs due to near 100% sequence identity surrounding those SNPs between the CD177 gene and CD177 pseudogene. Therefore, a modified TaqMan assay was necessary for CD177 exon 7 SNPs to achieve the gene-specificity in TaqMan assay. Genomic DNA was substituted with the CD177 gene-specific PCR fragment as the template for the TaqMan assay to determine CD177 ORF/STP genotypes. The CD177 gene-specific fragment containing ORF/STP variants was generated with gene-specific PCR. The sense primer (5′-ATT ATG ACA CAC GGA AAC TTG GCT C-3′ (SEQ ID NO:12)) and the antisense primer (5′-GTC CAA GGC CAT TAG GTT ATG AGG TCA GA-3′ (SEQ ID NO:15)) were used to amplify a CD177-specific genomic DNA fragment (2110 bps) (Table 1), which was subsequently used as the template for nested-PCR based TaqMan assay to determine the genotypes of CD177 variants. TaqMan genotyping assays of the ORF and STP haplotypes were carried out according to the standard protocol on an ABI 7500 Real-Time PCR System using Genotyping Master Mix (Applied Biosystems) with the sense primer (5′-CAC CCT CAG GAC TCA CAT CAA C-3′ (SEQ ID NO:38)), the antisense primer (5′-TGG TGG TCT TCT GGG AAT TTT G-3′ (SEQ ID NO:39)), the FAM-6 labeled STP allele probe (5′-FAM-TGG CGA CCT AAA G-MGBNFQ-3′ (SEQ ID NO:40)), and the VIC-labeled ORF allele probe (ORF: 5′-VIC-ACA AAA GGC TGC AGC AC-MGBNFQ-3′ (SEQ ID NO:41)).

### Example 12

### Statistical Analysis

The ANOVA and the nonparametric t-test (Mann-Whitney test) were used to determine whether HNA-2 positive cell population sizes and the HNA-2 deficiency are statistically associated with the nonsense CD177 cSNPs. The χ² test was used to determine whether observed genotype frequencies are consistent with Hardy-Weinberg equilibrium.

### Example 13

### Heterogeneous CD177 Expression on Blood Donor Neutrophils

As shown in FIG. 1B, the percentages of neutrophils expressing CD177 (or HNA-2) were heterogeneous among normal healthy blood donors in flow cytometry analysis. In 294 normal healthy blood donors, the percentage of CD177⁺ neutrophils ranged from 0.4% to 97.8%. Among 294 blood donors, we have identified 11 donors (or 3.7%) deficient for HNA-2 (less than 5% of granulocytes positive for CD177 mAb staining) and the percentage of HNA-2 deficient blood donors is consistent with those previously reported.

### Example 14

### Copy Number Variations (CNVs) of CD177 Gene

Copy number variations (CNVs) are the primary cause of HNA-1 (FcγRIIIB) deficiency and expression variations. To investigate whether CD177 CNVs are involved in HNA-2 deficiency, we have determined CD177 CNVs using TaqMan CNV assay kit Hs01327659_cn with the probe targeting the unique CD177 exon 1 region (FIG. 1A). Among 294 human subjects, 95.2% (280/294) subjects contained two-copy CD177 and 4.8% (14/294) were CD177 three-copy carriers. No human subjects had CD177 gene deletions among 294 subjects. Notably, all 11 HNA-2 deficient donors as identified in the flow cytometry assay were two-copy of CD gene carriers. In addition, all 11 HNA-2 deficient donors produced the full-length CD177 mRNAs as demonstrated by RT-PCR (FIG. 1C). This data clearly demonstrated that CD177 gene deletion (CNVs) and the lack of mRNA expression are not the cause of HNA-2 deficiency.

### Example 15

### Detection of a Novel Nonsense CD177 cSNP

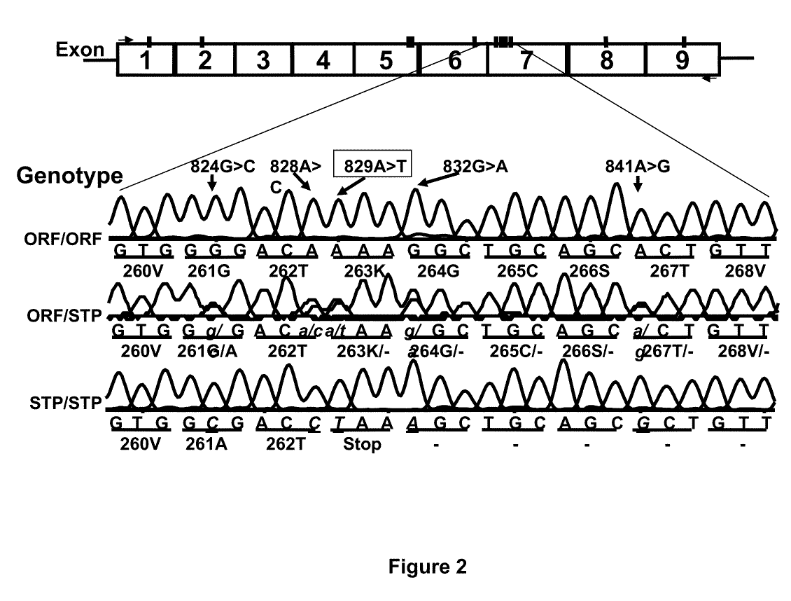

CD177 cDNA samples from those 11 HNA-2 deficient donors and 119 blood donors whose neutrophils express CD177 were subsequently sequenced. In addition to CD177 coding SNPs (cSNPs) identified previously, five novel cSNPs have been discovered (SNP 824G>C, 828A>C, 829A>T, 832G>A, and 841A>G) (Table 2), which form two haplotypes. Most importantly, the CD177 cSNP 829A>T is a nonsense polymorphism that creates a translation stop codon at amino acid position 263 (Lysine→Stop codon change) in the CD177 open reading frame (FIG. 2). Consequently, those two haplotypes were designated as the open reading haplotype (or ORF: 824G/828A/829A/832G/841A) and the stop codon haplotype (or STP haplotype: 824C/828C/829T/832A/841G). To determine the origin of the novel CD177 cSNP haplotype, we have also sequenced CD177 genomic DNA PCR products. Based on genomic DNA sequencing data, 72.1% (212/294) of donors were homozygous 829A donors and the homozygous 829T donors accounted for 3.1% (9/294) in our study population. The minor allele (829T) frequency was 15.5% (Table 3). The distribution of SNP 829A>T genotypes was consistent with the Hardy-Weinberg equilibrium in 294 blood donors (χ²=0.76, P=0.38) (Table 3).

### Example 16

### Association of the SNP 829A>T Genotypes with the CD177 Deficiency and Expression Variations

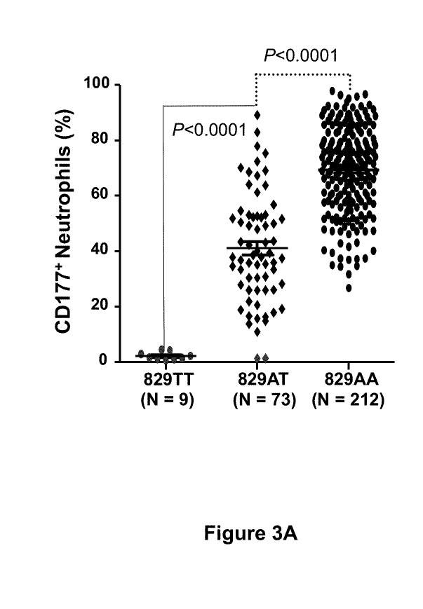

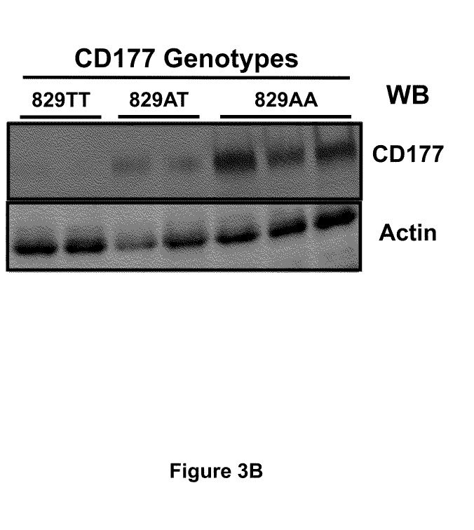

To examine whether the SNP 829A>T affect CD177 expression, the genotypes of all 294 phenotyped donors was determined through genomic DNA sequence analysis. As shown in FIG. 3A, all 829T homozygous donors were negative for neutrophil CD177 expression in flow cytometry analysis. In addition, the percentages of CD177⁺ neutrophils from the heterozygous donors (829AT) were significantly lower than those from homozygous 829A donors (P<0.0001). Western blot analyses also confirmed the absence of CD177 expression in 829TT homozygous donors and significantly less CD177 being expressed in the 829AT donors as compared to the 829AA homozygous donors (FIG. 3B). This data support the notion that the SNP 829A>T allele is a crucial determinant for CD177 deficiency and expression variations.

### Example 17

### Identification of a Rare Mutation in CD177 Deficient Donors

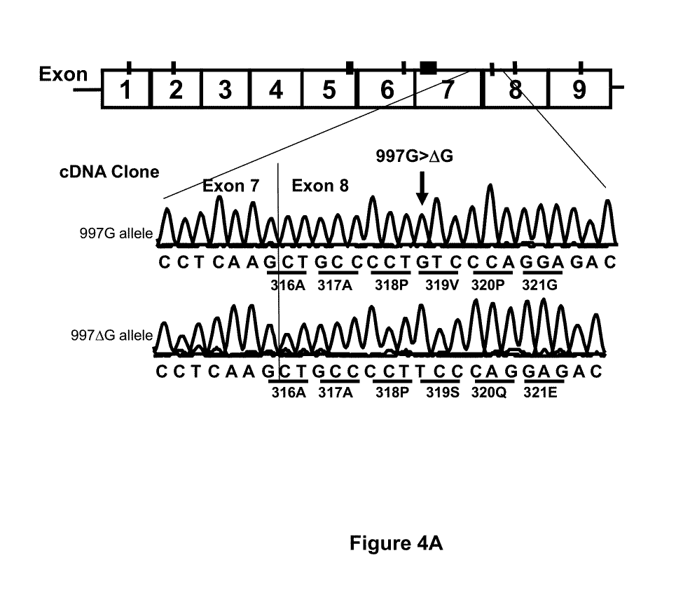

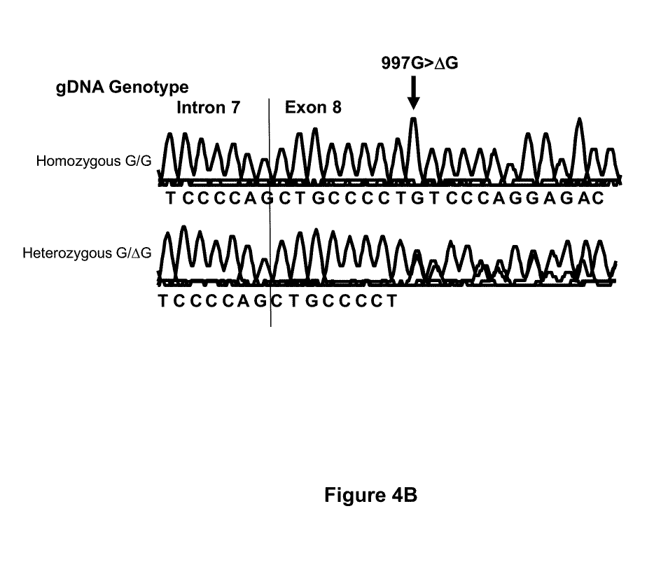

Although all homozygous 829TT donors (N=9) were negative for CD177 expression, two 829AT heterozygous donors were also negative for CD177 expression (FIG. 3A, donors in red color). The CD177 cDNAs from those two SNP 829A>T heterozygous donors were subsequently sequenced. Direct sequencing cDNAs revealed those two CD177⁻ donors heterozygous at SNP 829A>T had a heterozygous deletion of guanidine nucleotide at position 997. To confirm the deletion mutation at position 997, we cloned and sequenced cDNA from two CD177⁻ donors. As shown in FIG. 4A, two species of mRNAs were found in those two donors. The SNP 829T allele is in the linkage disequilibrium with wild-type at position 997 while the 829A allele (ORF allele) carries the guanidine deletion mutation at position 997. Genomic DNA sequence analysis confirmed that the guanidine nucleotide deletion occurs at genomic level (FIG. 4B). Our data indicate that the presence of the 829T allele in combination with the deletion mutation at position 997 on another chromosome could also lead to the CD177 expression deficiency. We found that only two out of 294 blood donors carry the guanidine deletion mutation on one chromosome by genomic sequencing analysis. Therefore, the allele frequency of the 997G deletion mutation is estimated to be 0.0034 in the current study population.

### Example 18

### Effect of CD177 cSNPs on CD177 Expression and Alloantibody Binding

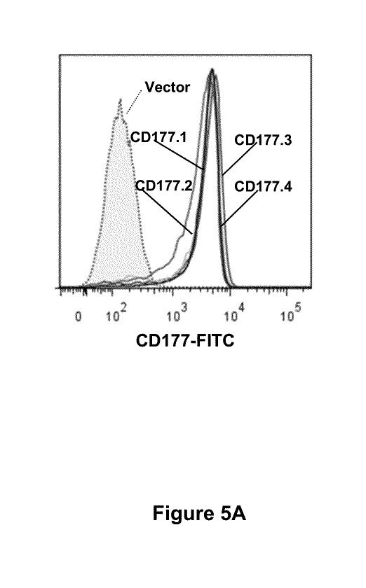

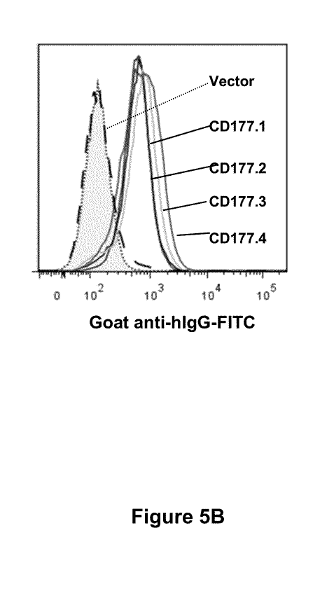

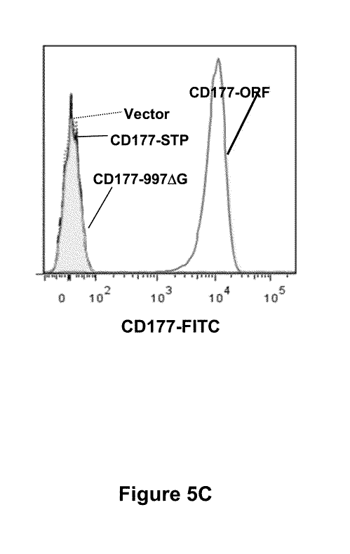

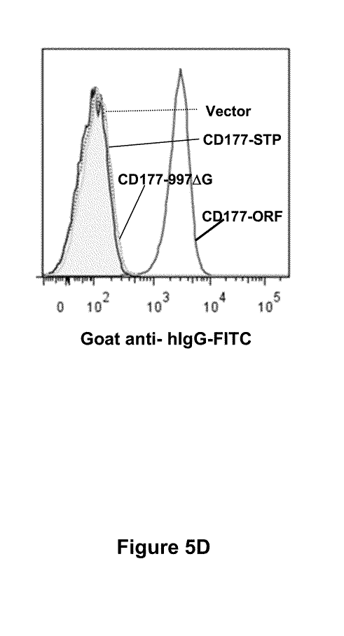

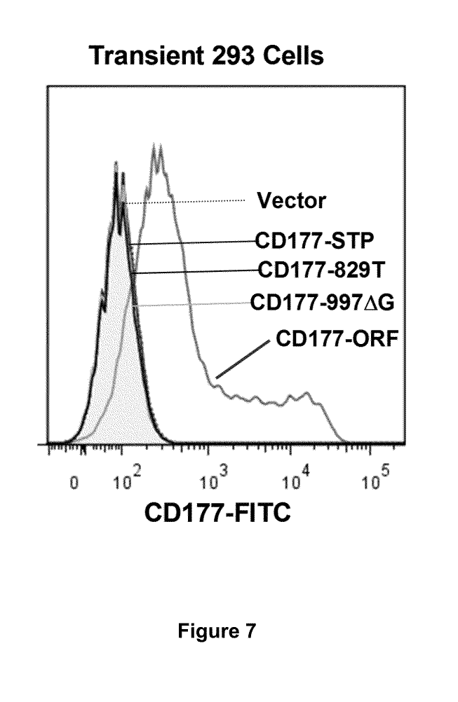

Although the genotypes of CD177 non-synonymous SNPs were reportedly associated with CD177 expression variations in genetic analyses, no experiment data were available about the effect of CD177 cSPNs on CD177 expression. To examine whether CD177 cSNPs affect the CD177 expression and the binding of HNA-2 alloantibodies, we have cloned the full-length CD177 cDNA variants containing the common non-conservative cSNPs (SNP 134A>T, 652A>G, 656G>T, and 1084G>A) in the CD177 mature peptide (aa22-408). There were no significant differences in the expression of CD177 (FIG. 5A) or in the binding to HNA-2 alloantibodies among four CD177 variants containing non-conservative amino acid substitutions (31His>Leu, 204Asn>Asp, 205Arg>Met, and 348Ala>Thr) (FIG. 5B), suggesting that non-synonymous CD177 cSNPs may not be responsible for the HNA-2 alloantibody production and expression variations. However, the CD177 variants carrying STP haplotype (CD177-STP) and the guanidine deletion at position 997 (CD177-997AG) failed to express CD177 on cell surface experiments (FIG. 5C) and had no reactivity with HNA-2 alloantibodies (FIG. 5D). Our data confirmed that both STP allele and 997G deletion mutation led to the CD177 expression deficiency. To pinpoint whether the nonsense SNP 829A>T in the STP haplotype is the key factor controlling CD177 expression, we generated a CD177 expression construct carrying the sole change at SNP 829A>T position. The T nucleotide substitution at position 829 alone led to the absence of CD177 expression in transfection experiments (FIG. 7), confirming that the SNP 829A>T is the sole determinant for CD177 expression in the STP haplotype.

Interestingly, it was found that all heterozygous donors of the SNP 829A>T in genomic DNA analysis primarily produced the SNP 829A allele (or ORF allele) mRNA based on their cDNA sequences. The nonsense SNP 829T allele tracer peak barely above the background was normally considered as sequence noises in the cDNA sequence analysis for heterozygous donors. This observation indicates that the CD177 mRNAs containing the nonsense 829T allele are not stable compared to the CD177 mRNAs containing the common 829A allele in the same donor, which may explain the failure in detecting the SNP 829A>T with cDNA sequencing strategy and the observation of the associations between certain CD177 cSNPs and the expression variations in previous studies.

### Example 19

### Development of CD177 SNP Genotyping Assays

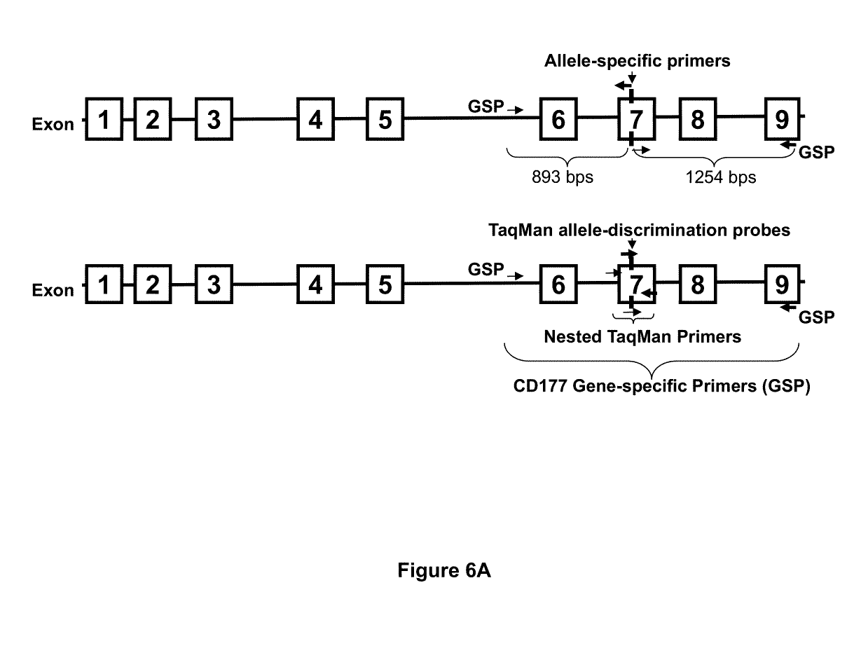

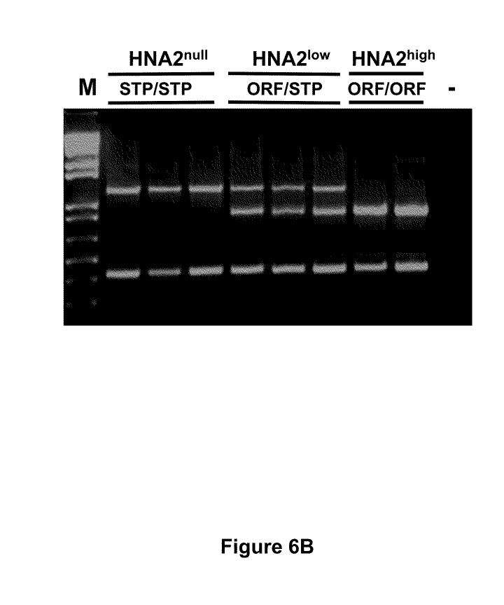

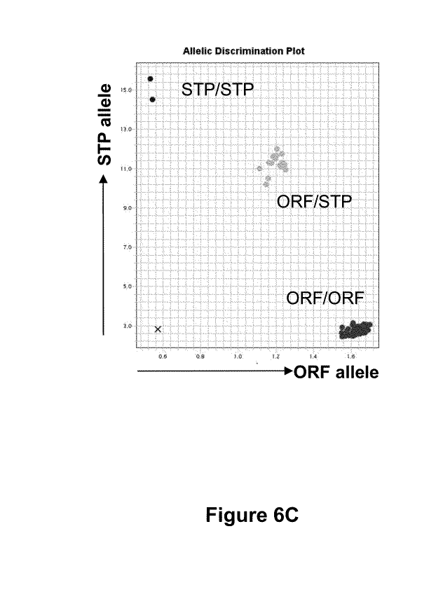

To facilitate genetic tests for HNA-2 system, primers were designed for a single-reaction allele-specific PCR assay to determine the genotypes of the CD177 SNP ORF or STP haplotypes (FIG. 6A, upper panel). As shown in FIG. 6B, the internal control of human growth hormone gene produced a DNA fragment of 429 bps. The ORF-allele generated a DNA fragment of 893 bps while the STP-allele produced a DNA fragment of 1254 bps. The specificity and accuracy of the assay were validated by the perfect match (100%) with 294 human subjects genotyped by Sanger sequencing methodology.

A high-throughput TaqMan assay based on nested-PCR was also developed to determine the genotypes of the CD177 ORF or STP variants (FIG. 6A, lower panel). As shown in FIG. 6C, the CD177 genotypes of STP/ORF variant are clustered in three populations. The STP homozygous genotype is on the upper left corner, the ORF/STP heterozygous genotype in the middle, and the ORF/ORF homozygous on the lower right corner. The genotypes obtained by TaqMan assay were subsequently compared to those determined by Sanger sequencing methodology. A perfect match (100%) between TaqMan assay and direct sequencing analysis was achieved in 294 human subjects, confirming the specificity and accuracy of the current TaqMan assay.

TABLE 1
Primers and probes for CD177 genetic analyses

| | | | DNA |
|---|---|---|---|
| | | SEQ ID | Size |
| Reactions | Primer Sequences (5′→3′) | NO | (bps) |
| RT-PCR | F: CTG AAA AAG CAG AAA GAG ATT ACC AGC CAC AG | 1 | 1411 |
| | R: GTC CAA GGC CAT TAG GTT ATG AGG TCA GA | 2 | |
| cDNA Sequencing | F1: A CCT TGA GGT GCC CAG TCT GCT T | 3 | — |
| | F2: ACC GGC AGT GTC CTA CCT GTG T | 4 | |
| | R1: AAG CAG ACT GGG CAC CTC AAG G | 5 | |
| | R2: ACA CAG GTA GGA CAC TGC CGG T | 6 | |
| gDNA Long-PCR | F: CTG AAA AAG CAG AAA GAG ATT ACC AGC CAC AG | 7 | 8728 |
| | R: GTC CAA GGC CAT TAG GTT ATG AGG TCA GA | 8 | |
| gDNA Short-PCR | F: ATT ATG ACA CAC GGA AAC TTG GCT C | 9 | 2110 |
| | R: GTC CAA GGC CAT TAG GTT ATG AGG TCA GA | 10 | |
| gDNA Sequencing | F: TCT TTG CCC CAC ACT AAA CA | 11 | — |
| ORF-Specific | F: ATT ATG ACA CAC GGA AAC TTG GCT C | 12 | 893 |
| | R: AAC AGT GCT GCA GCC TTT TGT CC | 13 | |
| STP-Specific | F: ATC AAC CCT GGT GGC GAC CTA AA | 14 | 1254 |
| | R: GTC CAA GGC CAT TAG GTT ATG AGG TCA GA | 15 | |
| SNP 997G- | F: GCT CCC CAG CTG CCC TTG T | 16 | 997 |
| Specific | R: GTC CAA GGC CAT TAG GTT ATG AGG TCA GA | 17 | |
| SNP 997ΔG- | F: TGC TCC CCA GCT GCC TCT T | 18 | 997 |
| Specific | R: GTC CAA GGC CAT TAG GTT ATG AGG TCA GA | 19 | |
| hGH Internal | F: GCC TTC CCA ACC ATT CCC | 20 | 429 |
| Control | R: TCA CGG ATT TCT GTT GTG TTT C | 21 | |

TABLE 2
SNPs in 130 donors cDNA region coding for the CD177
mature peptide

| | Nucleotide | Amino Acid | | Donors with |
|---|---|---|---|---|
| Exon | Substitution^{a} | Substitution^{b} | dbSNP# | minor allele |
| 2 | 134A > T | 31His > Leu | rs45553433 | 12/130 |
| 2 | 156G > A | 38Leu > Leu | rs45571738 | 15/130 |
| 5 | 593G > T | 184Gly > Val | rs71337594 | 6/130 |
| 5 | 652A > G | 204Asn > Asp | rs199668750 | 7/130 |
| 5 | 656G > T | 205Arg > Met | rs200662237 | 8/130 |
| 6 | 671C > T | 210Thr > Ile | rs182368720 | 1/130 |
| 6 | 782C > T | 247Thr > Met | rs148509022 | 3/130 |
| 6 | 793C > A | 251Leu > Ile | rs10425835 | 80/130 |
| 7 | 824G > C | 261Gly > Ala | Novel | 11/130^{c} |
| 7 | 828A > C | 262Thr > Thr | Novel | 11/130^{c} |
| 7 | 829A > T | 263Lys > Stop | Novel | 11/130^{c} |
| 7 | 832G > A | 264Gly > Ser | Novel | 11/130^{c} |
| 7 | 841A > G | 267Thr > Ala | Novel | 11/130^{c} |
| 8 | 1084G > A | 348Ala > Thr | rs61625631 | 48/130 |
| 9 | 1333G > A | 431Gly > Arg | rs78718189 | 21/130 |

^{a}Nucleotide position is relative to SEQ ID NO: 42
^{b}Amino Acid Position is counted from the ATG start codon
^{c}Among 11 HNA-2 deficient donors, nine donors are homozygous for the rare alleles and two are heterozygous of rare and common allele.

TABLE 3
Distribution of SNP 829A > T and summary of CD177
expression in blood donors

| | Human Subjects (N = 294) | CD177 Expression* |
|---|---|---|
| Genotype | | |
| AA (%) | 212 (72.1%) | High |
| AT (%) | 73 (24.8%) | Low-Medium |
| TT (%) | 9 (3.1%) | Negative |
| Allele | | |
| A (%) | 497 (84.5) | Yes |
| T (%) | 91 (15.5) | No |

*CD177 expression results were based on results of flow cytometry, Western blot, and transfection experiments.

TABLE 4
Mutagenesis primers for generation of CD177 expression constructs

| | | SEQ |
|---|---|---|
| | | ID |
| Nucleotide Change^{a} | Primer Sequences (5′→3′)^{b} | NO: |
| 134A > T | F: GACAGTTCAGC [symbol] TGTTGTGA | 22 |
| | R: TCGACACA [symbol] GCTGAACTGTC | 23 |
| 652A/656G > 652G/656T | F: ACTGAGAACTGC [symbol] ATA [symbol] GAAAGATTTTCTG | 24 |
| | R: CAGAAAATCTTTC [symbol] TAT [symbol] GCAGTTCTCAGT | 25 |
| 1084G > A | F: CCCAGGGGC [symbol] CCACTCATTGT | 26 |
| | R: ACAATGAGTGG [symbol] GCCCCTGG | 27 |
| 824G/828A/829A/832G/841A > | F: ACCCTGGTGG [symbol] GAC [symbol] AA [symbol] GCTGCAGC [symbol] CTGTTGGGGCT | 28 |
| 824C/828C/829T/832A/841G | R: AGCCCCAACAG [symbol] GCTGCAGC [symbol] TT [symbol] GTC [symbol] CCACCAGGGT | 29 |
| 829A > T | F: GTGGGGACA [symbol] AAGGCTGCAGCA | 30 |
| | R: TGCTGCAGCCTT [symbol] TGTCCCCAC | 31 |
| 997G > 997ΔG | F: CCTCAAGCTGCCCCT [symbol] TCCCAGGAGACCGG | 32 |
| | R: CCGGTCTCCTGGGA [symbol] AGGGGCAGCTTGAGG | 33 |

^{a}Nucleotide Position is relative to SEQ ID NO: 42
^{b}The target nucleotide mutation is highlighted with bold, italic, and underline.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of determining the CD177 haplotype of an individual, comprising:
providing a biological sample from the individual;
amplifying a CD177 nucleic acid in the biological sample using a pair of primer oligonucleotides to produce a CD177 amplification product, wherein the pair of primer oligonucleotides comprises SEQ ID NOs:9 and 10;
amplifying the CD177 amplification product with a pair of ORF-specific primer oligonucleotides comprising SEQ ID NOs:12 and 13 to produce an ORF amplification product of 893 bp when the ORF haplotype is present;
amplifying the CD177 amplification product with a pair of STP-specific primer oligonucleotides comprising SEQ ID NOs: 14 and 15 to produce a STP amplification product of 1254 bp when the STP haplotype is present;
thereby determining the CD177 haplotype of the individual.

2. The method of claim 1, wherein the individual is homozygous for the open reading haplotype (ORF) or homozygous for the stop codon haplotype (STP).

3. The method of claim 1, wherein the individual is heterozygous.

4. The method of claim 1, wherein an individual comprising a stop codon haplotype is prone to HNA-2 antigen formation.

5. The method of claim 1, wherein an individual comprising a stop codon haplotype exhibits HNA-2 deficiency.

6. The method of claim 1, wherein an individual comprising a stop codon haplotype is prone to produce alloantibodies responsible for transfusion related acute lung injury, bone marrow transplantation failure, neonatal neutropenia, alloimmune neutropenia, autoimmune neutropenia, bone marrow transplantation failure, and drug-induced immune neutropenia.

7. The method of claim 1, further comprising determining the nucleotide at position 997, numbered relative to SEQ ID NO:42.

8. The method of claim 7, wherein a wild type guanine at position 997 is in linkage disequilibrium with stop codon haplotype and wherein deletion of the guanine at position 997 is in linkage disequilibrium with the open reading haplotype.

9. The method of claim 1, wherein the biological sample is selected from the group consisting of blood, plasma, saliva, urine, epithelial cells, hair, bone marrow cells, biopsy tissues.

10. A method of determining the CD177 haplotype of an individual, comprising:
providing a biological sample from the individual;
amplifying a CD177 nucleic acid using at least one primer oligonucleotide in the presence of at least one probe oligonucleotide, wherein the at least one primer oligonucleotide is selected from the group consisting of SEQ ID NO:38 and SEQ ID NO:39, wherein the at least one probe oligonucleotide is selected from the group consisting of SEQ ID NO:40 and SEQ ID NO:41, wherein the at least one probe oligonucleotide is labeled with a first fluorescent moiety and a quencher moiety;
detecting the presence or absence of fluorescence emitted from the first fluorescent moiety,
thereby determining the CD177 haplotype of the individual.

11. The method of claim 10, wherein fluorescence in the presence of the probe oligonucleotide comprising SEQ ID NO:40 indicates the stop codon haplotype and wherein fluorescence in the presence of the probe oligonucleotide comprising SEQ ID NO:41 indicates the open reading haplotype.

12. The method of claim 10, wherein an individual comprising a stop codon haplotype is prone to HNA-2 antigen formation.

13. The method of claim 10, wherein an individual comprising a stop codon haplotype exhibits HNA-2 deficiency.

14. The method of claim 10, wherein an individual comprising a stop codon haplotype is prone to prone to produce alloantibodies responsible for transfusion related acute lung injury, bone marrow transplantation failure, neonatal neutropenia, alloimmune neutropenia, autoimmune neutropenia, bone marrow transplantation failure, and drug-induced immune neutropenia.

15. The method of claim 10, further comprising determining the nucleotide at position 997, numbered relative to SEQ ID NO:42.

16. The method of claim 15, wherein a wild type guanine at position 997 is in linkage disequilibrium with stop codon haplotype and wherein deletion of the guanine at position 997 is in linkage disequilibrium with the open reading haplotype.

17. The method of claim 10, wherein the biological sample is selected from the group consisting of blood, plasma, saliva, urine, epithelial cells, hair, bone marrow cells, biopsy tissues.


SEQUENCE LISTING (43 sequences)

SEQ ID NO: 1 - DNA, 32 bp, Artificial Sequence
  synthetic oligonucleotide
ctgaaaaagcagaaagagattaccagccacag

SEQ ID NO: 2 - DNA, 29 bp, Artificial Sequence
  synthetic oligonucleotide
gtccaaggccattaggttatgaggtcaga

SEQ ID NO: 3 - DNA, 23 bp, Artificial Sequence
  synthetic oligonucleotide
accttgaggtgcccagtctgctt

SEQ ID NO: 4 - DNA, 22 bp, Artificial Sequence
  synthetic oligonucleotide
accggcagtgtcctacctgtgt

SEQ ID NO: 5 - DNA, 22 bp, Artificial Sequence
  synthetic oligonucleotide
aagcagactgggcacctcaagg

SEQ ID NO: 6 - DNA, 22 bp, Artificial Sequence
  synthetic oligonucleotide
acacaggtaggacactgccggt

SEQ ID NO: 7 - DNA, 32 bp, Artificial Sequence; identical to SEQ ID NO: 1
  synthetic oligonucleotide

SEQ ID NO: 8 - DNA, 29 bp, Artificial Sequence
  synthetic oligonucleotide
gtccaaggccattaggttatgaggtcaga

SEQ ID NO: 9 - DNA, 25 bp, Artificial Sequence
  synthetic oligonucleotide
attatgacacacggaaacttggctc

SEQ ID NO: 10 - DNA, 29 bp, Artificial Sequence
  synthetic oligonucleotide
gtccaaggccattaggttatgaggtcaga

SEQ ID NO: 11 - DNA, 20 bp, Artificial Sequence
  synthetic oligonucleotide
tctttgccccacactaaaca

SEQ ID NO: 12 - DNA, 25 bp, Artificial Sequence
  synthetic oligonucleotide
attatgacacacggaaacttggctc

SEQ ID NO: 13 - DNA, 23 bp, Artificial Sequence
  synthetic oligonucleotide
aacagtgctgcagccttttgtcc

SEQ ID NO: 14 - DNA, 23 bp, Artificial Sequence
  synthetic oligonucleotide
atcaaccctggtggcgacctaaa

SEQ ID NO: 15 - DNA, 29 bp, Artificial Sequence
  synthetic oligonucleotide
gtccaaggccattaggttatgaggtcaga

SEQ ID NO: 16 - DNA, 19 bp, Artificial Sequence
  synthetic oligonucleotide
gctccccagctgcccttgt

SEQ ID NO: 17 - DNA, 29 bp, Artificial Sequence
  synthetic oligonucleotide
gtccaaggccattaggttatgaggtcaga

SEQ ID NO: 18 - DNA, 19 bp, Artificial Sequence
  synthetic oligonucleotide
tgctccccagctgcctctt

SEQ ID NO: 19 - DNA, 29 bp, Artificial Sequence
  synthetic oligonucleotide
gtccaaggccattaggttatgaggtcaga

SEQ ID NO: 20 - DNA, 18 bp, Artificial Sequence
  synthetic oligonucleotide
gccttcccaaccattccc

SEQ ID NO: 21 - DNA, 22 bp, Artificial Sequence
  synthetic oligonucleotide
tcacggatttctgttgtgtttc

SEQ ID NO: 22 - DNA, 20 bp, Artificial Sequence
  synthetic oligonucleotide
gacagttcagcttgttgtga

SEQ ID NO: 23 - DNA, 20 bp, Artificial Sequence
  synthetic oligonucleotide
tcgacacaagctgaactgtc

SEQ ID NO: 24 - DNA, 30 bp, Artificial Sequence
  synthetic oligonucleotide
actgagaactgcgatatgaaagattttctg

SEQ ID NO: 25 - DNA, 30 bp, Artificial Sequence
  synthetic oligonucleotide
cagaaaatctttcatatcgcagttctcagt

SEQ ID NO: 26 - DNA, 21 bp, Artificial Sequence
  synthetic oligonucleotide
cccaggggcgccactcattgt

SEQ ID NO: 27 - DNA, 20 bp, Artificial Sequence
  synthetic oligonucleotide
acaatgagtggcgcccctgg

SEQ ID NO: 28 - DNA, 39 bp, Artificial Sequence
  synthetic oligonucleotide
accctggtggcgacctaaagctgcagcgctgttggggct

SEQ ID NO: 29 - DNA, 39 bp, Artificial Sequence
  synthetic oligonucleotide
agccccaacagcgctgcagctttaggtcgccaccagggt

SEQ ID NO: 30 - DNA, 22 bp, Artificial Sequence
  synthetic oligonucleotide
gtggggacataaggctgcagca

SEQ ID NO: 31 - DNA, 22 bp, Artificial Sequence
  synthetic oligonucleotide
tgctgcagccttatgtccccac

SEQ ID NO: 32 - DNA, 29 bp, Artificial Sequence
  synthetic oligonucleotide
cctcaagctgccccttcccaggagaccgg

SEQ ID NO: 33 - DNA, 29 bp, Artificial Sequence
  synthetic oligonucleotide
ccggtctcctgggaaggggcagcttgagg

SEQ ID NO: 34 - DNA, 32 bp, Artificial Sequence
  synthetic oligonucleotide
cccaagcttaccagccacagacgggtcatgag

SEQ ID NO: 35 - DNA, 33 bp, Artificial Sequence
  synthetic oligonucleotide
tgctctagagaggtcagagggaggttgagtgtg

SEQ ID NO: 36 - DNA, 32 bp, Artificial Sequence
  synthetic oligonucleotide
cccggatccaccagccacagacgggtcatgag

SEQ ID NO: 37 - DNA, 33 bp, Artificial Sequence
  synthetic oligonucleotide
tgcgaattcgaggtcagagggaggttgagtgtg

SEQ ID NO: 38 - DNA, 22 bp, Artificial Sequence
  synthetic oligonucleotide
caccctcaggactcacatcaac

SEQ ID NO: 39 - DNA, 22 bp, Artificial Sequence
  synthetic oligonucleotide
tggtggtcttctgggaattttg

SEQ ID NO: 40 - DNA, 13 bp, Artificial Sequence
  synthetic oligonucleotide
tggcgacctaaag

SEQ ID NO: 41 - DNA, 17 bp, Artificial Sequence
  synthetic oligonucleotide
acaaaaggctgcagcac

SEQ ID NO: 42 - DNA, 2364 bp, Homo sapiens
ctgctgaaaaagcagaaagagattaccagccacagacgggtcatgagcgcggtattactgctggccctcctggggttcatcctcccactgccaggagtgcaggcgctgctctgccagtttgggacagttcagcatgtgtggaaggtgtccgacctgccccggcaatggacccctaagaacaccagctgcgacagcggcttggggtgccaggacacgttgatgctcattgagagcggaccccaagtgagcctggtgctctccaagggctgcacggaggccaaggaccaggagccccgcgtcactgagcaccggatgggccccggcctctccctgatctcctacaccttcgtgtgccgccaggaggacttctgcaacaacctcgttaactccctcccgctttgggccccacagcccccagcagacccaggatccttgaggtgcccagtctgcttgtctatggaaggctgtctggaggggacaacagaagagatctgccccaaggggaccacacactgttatgatggcctcctcaggctcaggggaggaggcatcttctccaatctgagagtccagggatgcatgccccagccagtttgcaacctgctcaatgggacacaggaaattgggcccgtgggtatgactgagaactgcgatatgaaagattttctgacctgtcatcgggggaccaccattatgacacacggaaacttggctcaagaacccactgattggaccacatcgaataccgagatgtgcgaggtggggcaggtgtgtcaggagacgctgctgctcctagatgtaggactcacatcaaccctggtggggacaaaaggctgcagcactgttggggctcaaaattcccagaagaccaccatccactcagcccctcctggggtgcttgtggcctcctatacccacttctgctcctcggacctgtgcaatagtgccagcagcagcagcgttctgctgaactccctccctcctcaagctgcccctgtcccaggagaccggcagtgtcctacctgtgtgcagccccttggaacctgttcaagtggctccccccgaatgacctgccccaggggcgccactcattgttatgatgggtacattcatctctcaggaggtgggctgtccaccaaaatgagcattcagggctgcgtggcccaaccttccagcttcttgttgaaccacaccagacaaatcgggatcttctctgcgcgtgagaagcgtgatgtgcagcctcctgcctctcagcatgagggaggtggggctgagggcctggagtctctcacttggggggtggggctggcactggccccagcgctgtggtggggagtggtttgcccttcctgctaactctattacccccacgattcttcaccgctgctgaccacccacactcaacctccctctgacctcataacctaatggccttggacaccagattctttcccattctgtccatgaatcatcttccccacacacaatcattcatatctactcacctaacagcaacactggggagagcctggagcatccggacttgccctatgggagaggggacgctggaggagtggctgcatgtatctgataatacagaccctgtcctttctcccagtgctgggatttctccatgtgagggggcagcaggacacccagggatctagcgtgggggaggagaggagcctaatgagaaaatgaccatctaaagcctgcccttcattggtctggttcacgtctccaaaccagcttggatggtagcagagacttcagggtgctccagccaaacgtatttgggcatcaccatgacctgggaggggaagatgcactgagacgtatgaggcttccagcctagcagccagggccctagcacaaacaggaggctcgccccatctgagcaactgcaggagaggttagtacagtcatgcattgcttaacgacagggacgtgtcgttagaaatgtgtcgttaggtgattttatgaccataggaacattgtagcgtgcacttacaccaacccagatggtacagcccaatacacacccaggatggacgctagagtcgactgctcctaggctacaagcctgcagtgcatgttatggtgtgaatactgcaggcaatcttaacaccacggcaagtatttgtgcatctacacacatctaaacatagaaaaggtacagcataaatacactattgtcatctcagcagaccaccgttctatacgcaattcgtcgctgacccaaacgttgctatgtagcatctgcgtatcgtgggataattgacatgagggcttgagagaactccagaaaaaaatgggttagcattttcccagagctgttatcattgggtctctcttaccaccata

SEQ ID NO: 43 - protein, 437 aa, Homo sapiens
MSAVLLLALLGFILPLPGVQALLCQFGTVQHVWKVSDLPRQWTPKNTSCDSGLGCQDTLMLIESGPQVSLVLSKGCTEAKDQEPRVTEHRMGPGLSLISYTFVCRQEDFCNNLVNSLPLWAPQPPADPGSLRCPVCLSMEGCLEGTTEEICPKGTTHCYDGLLRLRGGGIFSNLRVQGCMPQPVCNLLNGTQEIGPVGMTENCDMKDFLTCHRGTTIMTHGNLAQEPTDWTTSNTEMCEVGQVCQETLLLLDVGLTSTLVGTKGCSTVGAQNSQKTTIHSAPPGVLVASYTHFCSSDLCNSASSSSVLLNSLPPQAAPVPGDRQCPTCVQPLGTCSSGSPRMTCPRGATHCYDGYIHLSGGGLSTKMSIQGCVAQPSSFLLNHTRQIGIFSAREKRDVQPPASQHEGGGAEGLESLTWGVGLALAPALWWGVVCPSC